(19) United States
(12) Patent Application Publication
(10) Pub. No.: US 2013/0190719 A1
(43) Pub. Date: Jul. 25, 2013

(54) DRUG DELIVERY DEVICE WITH DRIVE MEMBER HAVING INDICATION ELEMENTS

(71) Applicants: Christopher James Smith, Cheshire (GB); Mark Philip Horlock, Cheshire (GB); Stephen David Butler, Staffordshire (GB)

(72) Inventors: Christopher James Smith, Cheshire (GB); Mark Philip Horlock, Cheshire (GB); Stephen David Butler, Staffordshire (GB)

(73) Assignee: Sanofi-Aventis Deutschland GmbH, Frankfurt am Main (DE)

(21) Appl. No.: 13/702,816

(22) PCT Filed: Jun. 9, 2011
(86) PCT No.: PCT/EP11/59573

§ 371 Date: Apr. 2, 2013

(30) Foreign Application Priority Data
Jun. 11, 2010 (EP) 10165644.5

(51) Int. Cl. A61M 5/315

(52) U.S. Cl. CPC A61M 5/31568
USPC 604/506; 604/189

(57) ABSTRACT

A drug delivery device is proposed, which comprises a housing, a drive member which is movably retained within the housing, a piston rod which is operatively coupled to the drive member, wherein the drug delivery device is configured to convert movement of the drive member with respect to the housing into movement of the piston rod with respect to the drive member, wherein a surface of the drive member is provided with one or a plurality of indication elements, and wherein the drug delivery device is adapted to display at least one of the indication elements through the housing. Furthermore it is proposed to use a single member in a drug delivery device for driving movement of a piston rod of the device and for displaying dose related information to a user of the device.

### CROSS REFERENCE TO RELATED APPLICATIONS

The present application is a U.S. National Phase Application pursuant to 35 U.S.C. §371 of International Application No. PCT/EP2011/059573 filed Jun. 9, 2011, which claims priority to European Patent Application No. 10165644.5 filed on Jun. 11, 2010. The entire disclosure contents of these applications are herewith incorporated by reference into the present application.

### FIELD OF INVENTION

The present disclosure relates to a drug delivery device and to the use of a member in such a device.

### BACKGROUND

Drug delivery devices are usually provided to administer a drug. Some of the devices are provided for self-administration of the drug by the user, i.e. without medically trained people being involved in the administration process. As the user may not be medically trained, the devices should be easy to use and reliably operable.

### SUMMARY

It is an object of the present disclosure to facilitate provision of a novel, preferably an improved device, in particular a device with improved operability by a user.

This object is achieved, for example, by the subject-matter of the independent claims. Further advantageous embodiments, refinements and developments are the subject-matter of the dependent claims.

An aspect of the present disclosure relates to a drug delivery device. The device comprises a housing and a drive member, which is moveably retained within the housing. A piston rod is operatively coupled to the drive member. The drug delivery device is configured to convert movement of the drive member with respect to the housing, preferably rotational movement of the drive member with respect to the housing, into movement of the piston rod. Preferably, the piston rod is moved with respect to the drive member. A surface of the drive member is provided with one or a plurality of indication elements. The drug delivery device is adapted to display at least one of the indication elements through the housing.

By providing indication elements on a surface of the drive member, preferably on an outer surface of the drive member, changes in the position of the drive member with respect to the housing, may be easily monitored by the user. As the drive member is coupled to the piston rod, the user may gather information about the position of the piston rod with respect to the housing via the indication element provided on the drive member. The piston rod is moved to dispense drug from the device. Thus, dose-related information may be displayed to the user by the indication elements, such as information on the number of doses which were already dispensed from the device or which remain to be dispensed from the device. Accordingly, the user may retrieve dose-related information immediately from a surface of the drive member on which the indication elements are provided.

Another aspect of the disclosure relates to the use of a single member in a drug delivery device for driving movement of a piston rod of the device and for displaying dose-related information to a user of the device. The single member is preferably unitary. Expediently, the single member corresponds to the drive member mentioned above. Accordingly, features described herein in connection with the drug delivery device may also relate to the use of the member and vice versa.

The drive member may be that member which immediately acts on the piston rod to cause movement of the piston rod with respect to the housing for dispensing a drug from the device.

Thus, a user may gather drug-related information from that member, which causes movement of the piston rod. Accordingly, as the drive member may be the last member which acts on the piston rod to cause the piston rod to be moved, information gathered from the drive member is very reliable. In particular, the information may be more reliable than the one derived from a separate dose indicator. Thus, a separate dose indicator, in addition to the drive member, may be dispensed with.

In an embodiment, the drive member and the piston rod are engaged. Engagement of drive member and piston rod facilitates a reliable coupling between drive member and piston rod to convert movement of the drive member into movement of the piston rod.

In another embodiment, the drive member is splined to the piston rod. A splined connection between drive member and piston rod facilitates conversion of rotation of the drive member into rotation of the piston rod.

In another embodiment, the device is a fixed dose device for dispensing a plurality of predefined doses of a drug. The size of the respective dose may not be varied by a user. All doses may have equal size.

In another embodiment, the displayed indication element provides information about the number of doses of drug dispensed from the device or remaining to be dispensed from the device.

In another embodiment, the drive member is configured to be rotatable with respect to the housing for dispensing a dose of drug. The rotation angle by which the drive member is rotated for dispensing the dose expediently corresponds to or is determined by the distance between adjacent indication elements.

In another embodiment, the device is configured such that the displayed indication element changes when a dose of drug is dispensed from the device. Particularly, by rotating the drive member, the displayed indication element may be substituted by another, in particular, an adjacent indication element.

In another embodiment, the housing has a proximal end and a distal end. The device is expediently configured to convert rotational movement of the drive member into distal movement of the piston rod for dispensing a dose of drug.

In another embodiment, for displaying the indication element, the housing may comprise a window. The window may frame the displayed indication element. Other indication elements which are not to be displayed may be hidden. Additionally or alternatively, the housing may be provided with a marking, e.g. a triangle or an arrow or the like or other means to distinguish the displayed indication element. This is particularly feasible if a plurality of indication elements may be viewed by the user through the housing. Instead of providing a separate window, a transparent housing may be used. However, as the mechanics in the housing may be viewed if a transparent housing is used, an opaque housing which is provided with a window, such as an aperture which may or may not be covered by a separate transparent window part, may be advantageous.

In another embodiment, in a resting state of the device, in which the device is not in use for setting or dispensing a dose, the drive member is secured against axial displacement with respect to the housing. Preferably, the window which is provided in the housing frames the displayed indication element. By securing the drive member against axial displacement in a resting state of the device, it may be ensured that the axial position of the displayed indication element is fixed, and the user may reliably retrieve information from this indication element.

In another embodiment, the drug delivery device comprises a rotation member which is adapted to be rotated with respect to the drive member in a first direction for setting a dose of drug, and to be rotated in a second direction with respect to the housing for dispensing the set dose. The rotation angle by which the rotation member is rotated for dose setting and/or for dose dispensing preferably corresponds to or is determined by the distance between adjacent indication elements. The rotation member may be rotated by the same angle in opposite directions for dose setting and dose dispensing.

In another embodiment, the drive member is coupled to the rotation member by a uni-directional clutch mechanism, in particular a friction clutch, which is, when the clutch mechanism is engaged, operative to permit relative rotational movement of the rotation member with respect to the drive member when the rotation member is rotated in the first direction and to prevent relative rotational movement of the rotation member with respect to the drive member when the rotation member is rotated in the second direction. Accordingly, the rotation member may carry the drive member with it when it is rotated in the second direction, when the clutch mechanism is engaged. Thus, the drive member may be rotated in the second direction for dispensing the set dose.

In another embodiment, the drug delivery device comprises a stop member. The stop member is expediently coupled to the drive member by a uni-directional clutch mechanism, in particular a friction clutch, which is, when the clutch mechanism is engaged, operative to prevent relative rotational movement of the drive member with respect to the stop member when the rotation member is rotated in the first direction and to permit relative rotational movement of the drive member with respect to the stop member in the second direction. Accordingly, rotational movement of the drive member with respect to the stop member during dose setting may be prevented, whereas relative movement of the drive member with respect to the stop member during dose dispensing may be allowed.

In another embodiment, the drug delivery device comprises a resilient member. The resilient member may be provided to keep the respective uni-directional clutch mechanism, i.e. the one between rotation member and drive member or the one between drive member and stop member, engaged when the device is operated for setting and dispensing a dose of drug. In particular, the drive member may be arranged between the stop member and the rotation member. The resilient member may be operative and arranged to keep the drive member in abutment with the stop member and the rotation member during dose setting and during dose dispensing. Thus, the clutch mechanism(s) may be in permanent engagement when the device is in an operating mode for setting and dispensing a drug. For switching the device from the operating mode into a reset mode, the respective clutch mechanism may be disengaged, thus allowing the drive member to be moved in a direction with respect to the housing which was previously not permitted. Accordingly, movement of the piston rod in a direction back towards an initial position, which usually requires a “reversed” movement of the drive member, may be allowed when the device is in the reset mode.

Further features, advantageous embodiments and refinements are described below in the description of the exemplary embodiments in conjunction with the accompanying drawings.

### BRIEF DESCRIPTION OF THE FIGURES

FIG. 1 schematically shows a partly sectional side view of an exemplary embodiment of a drug delivery device.

FIG. 2 schematically shows a perspective sectional view of a part of a drive mechanism according to a first embodiment with schematically indicated movements of elements thereof during setting of a dose.

FIG. 3 schematically shows a more detailed side view of a part of FIG. 2.

FIG. 4 schematically shows a perspective sectional view of a part of the drive mechanism according to the first embodiment with indicated movements of elements thereof during delivery of a dose.

FIG. 5 schematically shows a more detailed side view of a part of FIG. 4.

FIG. 6 schematically shows a perspective sectional view of a part of a drive mechanism that is configured in accordance with the first embodiment.

FIG. 7 schematically shows a perspective view of a part of the drive mechanism of FIG. 2 with indicated movements of elements thereof during delivery of a dose.

FIG. 8 schematically shows a perspective view of a part of a drive mechanism that is configured in accordance with the first embodiment.

FIG. 9 schematically shows a perspective view of a part of a drive mechanism that is configured in accordance with the first embodiment.

FIG. 10 schematically shows an oblique sectional view of an embodiment of a drive mechanism.

FIG. 11 shows a schematic sectional view of a part of a resettable drive mechanism according to an embodiment in delivery position.

FIG. 12 shows the resettable drive mechanism of FIG. 11 in reset position.

FIG. 13 shows parts of another exemplary embodiment of a drug delivery device on the basis of a schematic sectional view.

FIGS. 14A to 14C show another exemplary embodiment of a drug delivery device on the basis of a schematic perspective view in FIG. 14A, a schematic side view in FIG. 14B and a schematic sectional view in FIG. 14C.

### DETAILED DESCRIPTION

Like elements, elements of the same kind and identically acting elements may be provided with the same reference numerals in the figures.

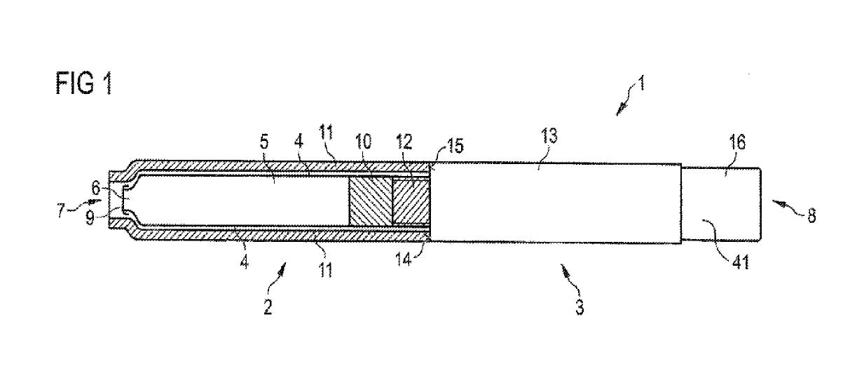

Turning now to FIG. 1, a drug delivery device 1 comprises a cartridge unit 2 and a drive unit 3. The cartridge unit 2 comprises a cartridge 4. Drug 5 is retained in the cartridge 4. The drug 5 is preferably liquid drug. The cartridge 4 preferably comprises a plurality of doses of the drug 5. The drug 5 may comprise insulin, heparin, or growth hormones, for example.

The term “drug”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(02)25, Asp28] Exendin-4(1-39),
des Pro36 [Met(O)14 Trp(02)25, IsoAsp28] Exendin-4(1-39),
   wherein the group-Lys6-NH2 may be bound to the C-terminus of the Exendin-4 derivative;
   or an Exendin-4 derivative of the sequence
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

The cartridge 4 has an outlet 6 at its distal end. Drug 5 can be dispensed from the cartridge through outlet 6. The device 1 may be a pen-type device, in particular a pen-type injector. The device 1 may be a disposable or a reusable device. The device 1 may be a device configured to dispense fixed doses of the drug or variable, preferably user-settable, doses. In a fixed dose device, the doses of drug which are to be dispensed may be preset, i.e. the user cannot vary the size of the dose. In a fixed dose device, the doses may have equal sizes. The device 1 may be a needle-based or a needle free device. The device 1 may be an injection device. The device may be a device for the self-administration of a drug by a user without formal medical training

The term “distal end” of the drug delivery device 1 or a component thereof may refer to that end of the device or the component which is closest to the dispensing end of the device 1. The term “proximal end” of the drug delivery device 1 or a component thereof may refer to that end of the device or the component which is furthest away from the dispensing end of the device. In FIG. 1, the distal end of the device 1 was assigned reference numeral 7 and the proximal end of the device was assigned reference numeral 8.

The outlet 6 may be covered by a membrane 9, which protects drug 5 against external influences during storage of the cartridge. For drug delivery, membrane 9 may be opened, e.g. pierced. For example, membrane 9 may be pierced by a needle unit (not explicitly shown). The needle unit may be (releasably) attached to the distal end of the cartridge unit 2. The needle unit may provide for fluid communication from the inside of the cartridge 4 to the outside of the cartridge through outlet 6.

A piston 10 is retained within the cartridge 4. The piston 10 is movable with respect to the cartridge. The piston 10 may seal the drug 5 within the cartridge. The piston 10 expediently seals the interior of the cartridge 4 proximally. Movement of the piston 10 with respect to the cartridge 4 in the distal direction causes drug 5 to be dispensed from the cartridge through outlet 6 during operation of the device.

The cartridge unit 2 furthermore comprises a cartridge retaining member 11. The cartridge 4 is retained within the cartridge retaining member 11. The cartridge retaining member 11 may stabilize the cartridge 4 mechanically. Additionally or alternatively, the cartridge retaining member 11 may be provided with a fixing member (not explicitly shown) for attaching the cartridge unit 2 to the drive unit 3.

The cartridge unit 2 and the drive unit 3 are secured to one another, preferably releasably secured. A cartridge unit 2 which is releasably secured to the drive unit may be detached from the drive unit 3, for example in order to allow for providing for a new cartridge 4, if all of the doses of drug which once were in the cartridge formerly attached to the drive unit 3 have already been dispensed. The cartridge retaining member 11 may be releasably secured to the drive unit 3 via a thread, for example.

Alternatively, the cartridge retaining member 11 may be dispensed with. It is particularly expedient, in this case, to apply a robust cartridge 4 and to attach the cartridge directly to the drive unit 3.

The drive unit 3 is configured for transferring force, preferably user-exerted force, particularly preferably manually exerted force, to the piston 10 for displacing the piston 10 with respect to the cartridge 4 in the distal direction. A dose of drug may be dispensed from the cartridge in this way. The size of the delivered dose may be determined by the distance by which the piston 10 is displaced with respect to the cartridge 4 in the distal direction.

The drive unit 3 comprises a drive mechanism. The drive mechanism comprises a piston rod 12. The piston rod 12 may be configured for transferring force to the piston 10, thereby displacing the piston in the distal direction with respect to the cartridge 4. A distal end face of the piston rod 12 may be arranged to abut a proximal end face of the piston 10. A bearing member (not explicitly shown) may be arranged to advance the piston 10, preferably to abut the proximal end face of the piston 10. The bearing member may be arranged between piston 10 and piston rod 12. The bearing member may be fixed to the piston rod 12 or a separate member. If the piston rod 12 is configured to be rotated during operation of the device, for example during dose delivery, it is particularly expedient to provide for a bearing member. The bearing member may be displaced together with the (rotating) piston rod with respect to the housing. The piston rod may be rotatable with respect to the bearing member. In this way, the risk that the rotating piston rod drills into the piston and thereby damages the piston is reduced. Accordingly, while the piston rod rotates and is displaced with respect to the housing, the bearing member is preferably only displaced, i.e. does not rotate. The piston rod may be bounded by the bearing member.

The drive unit 3 comprises a housing 13 which may be part of the drive mechanism. The piston rod 12 may be retained in the housing. A proximal end side 14 of the cartridge unit 2 may be secured to the drive unit 3 at a distal end side 15 of the housing 13, for example via a threaded connection. Housing 13, cartridge 4 and/or cartridge retaining member 11 may have a tubular shape.

The term “housing” shall preferably mean any exterior housing (“main housing”, “body”, “shell”) or interior housing (“insert”, “inner body”) which may have a unidirectional axial coupling to prevent proximal movement of specific components. The housing may be designed to enable the safe, correct, and comfortable handling of the drug delivery device or any of its mechanism. Usually, it is designed to house, fix, protect, guide, and/or engage with any of the inner components of the drug delivery device (e.g., the drive mechanism, cartridge, piston, piston rod), preferably by limiting the exposure to contaminants, such as liquid, dust, dirt etc. In general, the housing may be unitary or a multipart component of tubular or non-tubular shape.

The term “piston rod” shall preferably mean a component adapted to operate through/within the housing, which may be designed to transfer axial movement through/within the drug delivery device, preferably from the drive member to the piston, for example for the purpose of discharging/dispensing an injectable product. Said piston rod may be flexible or not. It may be a simple rod, a lead-screw, a rack and pinion system, a worm gear system, or the like. “Piston rod” shall further mean a component having a circular or non-circular cross-section. It may be made of any suitable material known by a person skilled in the art and may be of unitary or multipart construction.

The drive unit 3 comprises a dose part 16, e.g. a dose button. The dose part 16 is movable with respect to the housing 13. The dose part 16 may be movable in the proximal direction with respect to the housing for setting of a dose of the drug 5 which is to be delivered and in the distal direction with respect to the housing for delivery of the set dose. The dose part 16 is preferably connected to the housing 13. The dose part 16 may be secured against rotational movement with respect to the housing. The dose part 16 may be moved (displaced) between a proximal end position and a distal end position with respect to the housing 13 (not explicitly shown). The distance by which the dose part is displaced with respect to the housing during setting of the dose may determine a size of the dose. The proximal end position and the distal end position may be determined by a respective stop feature which may limit the proximal or distal travel of the dose member with respect to the housing. The device 1 may be a variable dose device, i.e. a device configured for delivering doses of drug of different, preferably user-settable, sizes. Alternatively, the device may be a fixed dose device.

The device 1 may be a manually, in particular non-electrically, driven device. The (user-applied) force which causes the dose part 16 to be moved with respect to the housing 13 in the distal direction may be transferred to the piston rod 12 by the drive mechanism. For this purpose, other elements of the drive mechanism may be provided which are not explicitly shown in FIG. 1. The drive mechanism is preferably configured to not move the piston rod 12 with respect to the housing 13 when the dose part is moved in the proximal direction with respect to the housing for setting of the dose.

Embodiments of a drive mechanism which are suitable to be provided in the drug delivery device 1 as it was described above are described in more detail below.

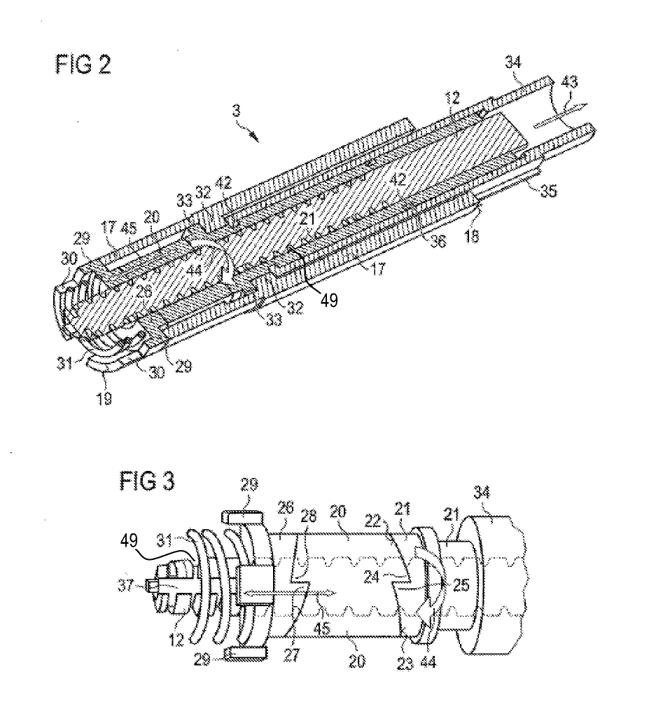

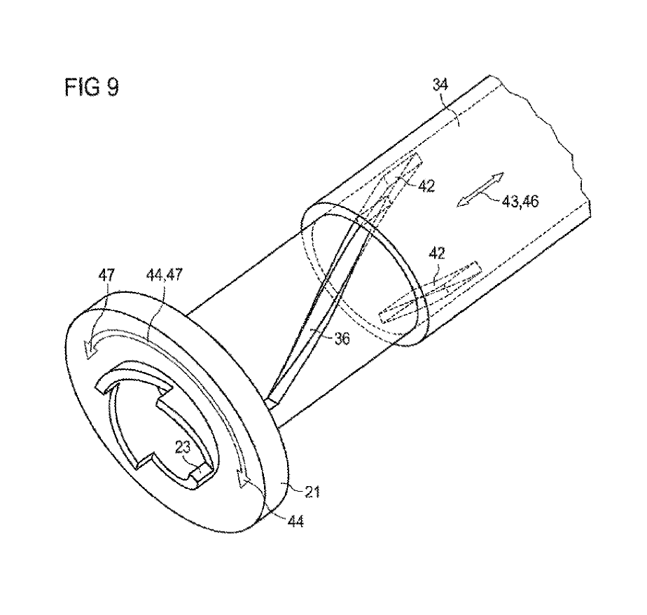

A first embodiment of a drive mechanism which is suitable for being implemented in the drug delivery device 1 as described above is described in connection with FIGS. 2 to 9.

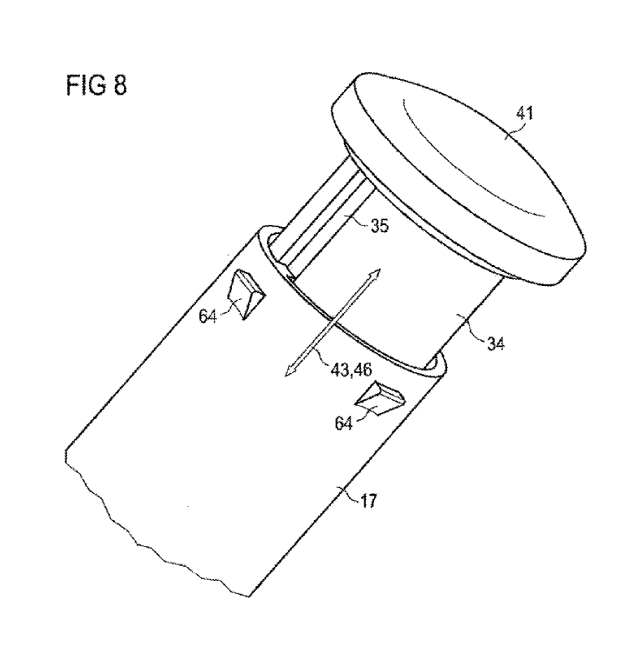

The drive mechanism comprises a housing part 17. The housing part 17 has a proximal end 18 and a distal end 19. The housing part 17 may be (outer) housing 13 of FIG. 1, a part thereof or an insert within housing 13, which insert is preferably secured against rotational and axial movement with respect to housing 13. The housing part 17 may be an insert sleeve, for example. The insert sleeve may be snap-fitted or glued to housing 13, for example. The housing part 17 may have a tubular shape. Housing part 17 may comprise outer fixing elements 64, for example snap-fit elements, for fixing housing part 17 to housing 13 (cf. FIG. 8).

The piston rod 12 is retained in the housing 13, preferably within housing part 17. The piston rod 12 is driven in the distal direction with respect to the housing part 17 during dose delivery.

The drive mechanism furthermore comprises a drive member 20. Drive member 20 is retained within the housing part 17. Drive member 20 is configured to transfer force, preferably torque, to the piston rod 12. The transferred force may cause the piston rod 12 to be displaced in the distal direction with respect to the housing part 17 for dose delivery.

Drive member 20 is rotatable with respect to housing part 17. The drive member 20 may engage the piston rod 12. Rotational movement of the drive member, for example rotational movement in a second direction may be converted into distal movement of the piston rod 12 with respect to the housing part 17. This is explained in more detail below.

The drive mechanism furthermore comprises a rotation member 21. The rotation member 21 is rotatable with respect to the housing part 17 in a first direction, in particular for setting of a dose of the drug, and in a second direction, in particular for delivering the set dose. The second direction is opposite to the first direction. The first direction may be counter-clockwise and the second direction may be clockwise as seen from the proximal end of the device, for example.

Drive member, rotation member and/or piston rod are preferably configured to be rotatable about a (common) rotation axis. The rotation axis may extend through drive member, rotation member and/or piston rod. The rotation axis may be the main longitudinal axis of the piston rod. The rotation axis may run between the proximal end and the distal end of the housing part 17.

The rotation member 21 is coupled to the drive member 20 by a uni-directional clutch mechanism, in particular a friction clutch mechanism. This clutch mechanism permits rotational movement of the rotation member 21 with respect to the drive member 20 when the rotation member rotates in the first direction with respect to the housing part 17. The clutch mechanism prevents rotational movement of the rotation member 21 with respect to the drive member 20, when the rotation member rotates in the second direction with respect to the housing part 17. The drive member 20 may thus follow rotational movement of the rotation member 21 in the second direction with respect to the housing part 17.

The drive member 20 is arranged to abut and/or engage the rotation member and, in particular, engages rotation member 21. The drive member 20 comprises a toothing 22. Toothing 22 may be provided at one end of the drive member, e.g. its proximal end. The rotation member comprises a toothing 23. Toothings 22 and 23 face one another. Toothing 23 may be provided at one end of the rotation member which end faces the drive member 20, e.g. at the distal end of the rotation member. Toothing 22 comprises a plurality of teeth 24. Toothing 23 comprises a plurality of teeth 25. Teeth 24 and/or 25 may extend and preferably may be oriented along the rotation axis. Toothings 22 and 23 may be configured to mate with one another. The rotation member and the drive member may engage each other by toothings 22 and 23 being in engagement.

A respective tooth of teeth 24 and/or teeth 25 may be ramp-shaped, in particular along the azimuthal (angular) direction as seen from the rotation axis. The ramp of the respective tooth is limited (in the angular direction) by a steep end face of that tooth, i.e. a face of the tooth that runs parallel to the rotation axis or includes a smaller angle with the rotation axis when projected on this axis than the ramp when projected on this axis. The steep end face is followed by the ramp of the next tooth.

The teeth 24 may be circumferentially disposed on the drive member 20, particularly at the end of the drive member 20 which faces the rotation member 21. The teeth 25 may be circumferentially disposed on the rotation member 21, particularly at the end of the rotation member 21 which faces the drive member 20.

When the steep end faces of two teeth abut and the rotation member is rotated further on in the second direction, the steep sides stay in abutment and drive member 20 follows the rotation of rotation member 21. When the rotation member rotates in the first direction, the ramp of the teeth—which ramps, in particular, run obliquely with respect to the rotation axis—slide along each other and, in consequence, the rotation member 21 may rotate with respect to the drive member 20.

The drive mechanism furthermore comprises a stop member 26. The drive member may be arranged between the stop member 26 and the rotation member 21. The stop member 26 is configured for preventing rotational movement of the drive member 20 in the first direction with respect to the housing part 17 during setting of a dose, i.e. when the rotation member rotates in the first direction. Thus, the rotation member 21 may rotate in the first direction with respect to the housing part 17, whereas the drive member 20 and the stop member 21 do not rotate.

The stop member 26 is coupled to the drive member 20 by another uni-directional clutch mechanism, in particular a friction clutch mechanism. This clutch mechanism prevents rotational movement of the drive member 20 with respect to the stop member 26 when the rotation member 21 rotates in the first direction with respect to the housing part 17. The clutch mechanism permits rotational movement of the drive member 20 with respect to the stop member 26, when the rotation member 21 rotates in the second direction with respect to the housing part 17.

Thus, the rotation member 21 may rotate with respect to the drive member 20 and the stop member 26 in the first direction during setting of the dose, with rotation of the drive member 20 being prevented by its interaction with the stop member 26, and rotation member 21 as well as drive member 20 may rotate with respect to the stop member 26 in the second direction during delivery of the dose.

The stop member 26 may be arranged to abut and/or engage the drive member 20 during setting of the dose and, preferably, during delivery of the dose. The stop member 26 has a toothing 27. Toothing 27 may be provided at one end of the stop member which faces the drive member, e.g. its proximal end. The teeth may be ramp-shaped with a steep side and a less steep ramp. The teeth may be azimuthally disposed along the stop member, in particular on the perimeter of the stop member. The teeth may extend and preferably may be oriented along the rotation axis.

Drive member 20 has a toothing 28. Toothing 28 may be provided at one end of the drive member which faces the stop member, e.g. the distal end of the drive member. The teeth of toothing 28 may extend and preferably may be oriented along the rotation axis. Toothings 22 and 28 of the drive member 20 are oppositely disposed. Toothing 28 may be configured in accordance with toothing 23 of the rotation member. Toothing 22 may be configured in accordance with toothing 27 of the stop member. Toothings 27 and 28 may face one another. Toothings 27 and 28 may mate with one another. Toothings 27 and 28, in particular the steep sides of the teeth, do cooperate, e.g. abut, for preventing rotation of the drive member 20 with respect to the housing part 17 and, in particular, with respect to the stop member 26 in the first direction.

Stop member 26 is preferably secured against rotational movement, particularly preferably permanently secured against rotational movement, with respect to the housing part 17. Stop member 26 may be fixed to the housing or integrated into the housing. Stop member 26 may be fixed against displacement with respect to the housing part 17 or displacement with respect to the housing part 17 may be allowed.

As it is illustrated in the present embodiment, stop member 26 is displaceable with respect to the housing but non-rotatable with respect to the housing part 17. For that purpose, one or a plurality of, preferably oppositely disposed, guide features, for example guide lugs 29, are provided in the stop member 26. The respective guide feature 29 engages a corresponding guide slot 30 which may be provided in the housing, e.g. in housing part 17. This can be seen in FIGS. 2 to 5. A guide feature 29 cooperates with a guide slot 30 to prevent rotational movement of the stop member with respect to the housing part 17, with axial movement of the stop member 26 with respect to the housing being allowed. The axial movement of the stop member 26 may compensate for play between components of the drive mechanism during operation.

From the group comprising drive member 20, stop member 26 and rotation member 21 one or more members, preferably two members or three members, may be axially displaceable with respect to the housing part 17 and, preferably, with respect to the piston rod 12. Therein, the drive member and another one of the recited members may be axially displaceable with respect to the housing. The remaining member may be secured against axial displacement or may also be axially displaceable during operation of the drive mechanism for drug delivery. Accordingly, if the drive member and the stop member are axially displaceable, the rotation member may be axially secured or axially displaceable and so on. Play between the components caused by relative (axial) movement of components of the clutch mechanism with respect to the housing can be compensated for in this way. The distance by which the respective components may be axially displaced with respect to the housing may correspond to the (maximum) depth of a tooth of the respective toothing 22 or 28 of the drive member. Alternatively, the distance may be greater than the (maximum) depth of a tooth of the respective toothing.

Furthermore, the drive mechanism comprises a resilient member 31, preferably a spring member. The resilient member 31 may be biased during drug delivery operation of the drive mechanism. The resilient member may provide for a force that tends to keep the drive member 20 in engagement with the stop member 26 and/or the rotation member 21. The force may be exerted along the rotation axis. In the situation shown in FIGS. 2 to 5, this force may be exerted in the proximal direction. The resilient member 31 may be a helical (coil) spring. The resilient member 31 may be a compression spring.

The resilient member 31 may keep the drive member 20 and the stop member 26 in (permanent) mechanical contact, e.g. in abutment, with each other during setting and delivery of a dose of the drug. Alternatively or additionally, the resilient member 31 may keep the drive member 20 and the rotation member 26 in (permanent) mechanical contact, preferably abutment, with each other during setting and delivery of a dose of the drug.

The resilient member 31 may be integrated within stop member 26 or a separate component. The resilient member 31 may be arranged on the distal end side of the stop member 26.

The drive mechanism furthermore comprises a support member 32. Support member 32 is expediently fixed against axial and rotational movement with respect to the housing part 17 or integrated into housing part 17. Support member 32 is arranged on that side of the drive member 20 which is remote from the stop member 26. Support member 32 may be a protrusion, for example a ring-like protrusion. Rotation member 21 may extend through an opening in support member 32. The support member 32 may provide for a counter force to the force which is exerted by the resilient member 31. Permanent abutment of the rotation member with the drive member and of the drive member with the stop member during setting and delivery of drug is facilitated in this way.

The rotation member 21 has an (radially) outwardly protruding member 33, for example a flange portion. The protruding member 33 is expediently provided for abutting support member 32, in particular the distal end side of support member 32.

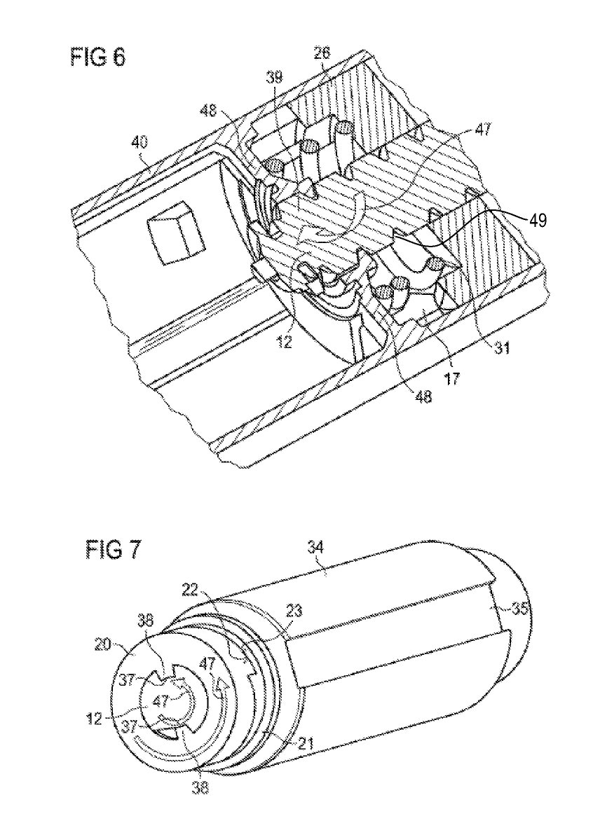

Another support 48 (cf. FIG. 6) may be provided for providing a counterforce to the force exerted by the resilient member 31. Support 48 is arranged on that side of the drive member 20 which is remote from the rotation member 21. Support 48 is arranged on that side of the stop member 26 which is remote from the support member 32. The support 48 may be arranged to abut the resilient member 31. The support 48 may be secured against axial and rotational movement with respect to the housing part 17, with respect to the housing 13 or integrated into the housing 13, for example into (additional) housing part 40 (cf. FIG. 6).

The drive mechanism furthermore comprises a dose member 34. Dose member 34 may be dose part 16 or may be a part of the dose part 16 of FIG. 1. Dose member 34 is movable with respect to the housing in the proximal direction for setting of a dose and for delivery of the dose. For example, the dose member 34 may be moved in the proximal direction with respect to the housing part 17 during dose setting and in the distal direction with respect to the housing part 17 during dose delivery. The dose member 34 may engage the housing part 17 or, alternatively, another part of housing 13 (not explicitly shown). Dose member 34 is preferably secured against rotational movement with respect to the housing part 17. The dose member 34 may comprise a guide feature 35, for example a guide lug or a guide slot, that engages another guide feature, for example a guide slot or a guide lug, respectively, that is provided in the housing part 17 or the housing 13. The dose member 34 may be displaced with respect to housing part 17 preferably only axially along and/or rotationally around the rotation axis.

Dose member 34 may be moved in the proximal direction and in the distal direction with respect to rotation member 21. Dose member 34 is arranged to be coupleable and is preferably (permanently) coupled to rotation member 21 such that movement of the dose member, e.g. in the proximal direction with respect to the housing part 17, for setting a dose of the drug is converted into rotational movement of the rotation member in the first direction and movement of the dose member, e.g. in the distal direction with respect to the housing part 17, for delivering the dose is converted into rotational movement of the rotation member 21 in the second direction opposite to the first direction.

The rotation member 21 may be provided with an (outer) thread 36. Thread 36 may be engaged with one of or a plurality of engagement members 42 of dose member 34. The respective engagement member may be arranged on the inside of the dose member. The respective engagement member may be a thread or a part of a thread, for example. Thus, dose member 34 and rotation member 21 may be threadedly coupled, in particularly threadedly engaged. The rotation member 21 may be arranged inside the dose member 21.

The rotation member 21, the drive member 20, the stop member 26 and/or the dose member 34 may be or may comprise a respective sleeve. The piston rod 12 may be arranged to be driven and, in particular, may be driven through one of, more of or all of those sleeves. The piston rod 12 may run through one of, more of or all of those sleeves.

The drive member 20 and the piston rod 12 are configured for rotational movement of the drive member 20 with respect to the housing being converted into rotational movement of the piston rod with respect to the housing. The drive member 20 may engage the piston rod 12. The piston rod 12 is displaceable with respect to the drive member 20 along a displacement axis. Presently, the displacement axis runs along the rotation axis. The drive member 20 may be splined to the piston rod 12, for example.

The piston rod 12 is threadedly coupled to the housing 13. The piston rod 12 may be provided with an outer thread 49, for example. The piston rod 12 may extend through and be engaged with a (part) thread in opening 39 which is provided in housing part 40, for example in support 48 (cf. FIG. 6). Housing part 40 may be formed integrally with housing part 17, may be a housing part fixed thereto or may be a housing part secured separately from housing part 17 to housing 13.

The piston rod 12 comprises an engagement track 37, preferably two oppositely disposed engagement tracks, on the outside. The (respective) engagement track 37 may interrupt thread 49. The (respective) engagement track 37 preferably extends along the axis along which the piston rod is displaceable with respect to the housing and, in particular, with respect to the drive member.

Rotational movement of the drive member 20 with respect to the housing may thus be converted into rotational movement of the piston rod 12 with respect to the housing and the rotational movement of the piston rod 12 is, on account of the threaded engagement of the piston rod and the housing (part), converted into movement of the piston rod with respect to the housing in the distal direction.

The dose part 16 (cf. FIG. 1) may comprise a dose button 41 (cf. FIG. 8). Dose button 41 may be configured to be gripped by a user. Dose button 41 may be arranged and connected to the dose member 34 at the proximal end. Dose button and dose member may be unitary.

In the following, operation of the present drive mechanism for delivering drug from the cartridge 4 of FIG. 1 is described.

To set a dose, a user may manually move dose member 34 in the proximal direction (arrow 43) with respect to the housing part 17 (cf. FIGS. 2, 3, 8 and 9). To do so, the user may grip dose button 41 and pull it in the proximal direction. Dose member 34 moves proximally also with respect to the rotation member 21. Proximal movement of the rotation member is prevented by support member 32 which abuts protruding member 33 of rotation member 21. Consequently, the proximal movement of dose member 34 with respect to the housing part 17 is converted into rotational movement of the rotation member 21 in the first direction (arrow 44) with respect to the housing part 17, in particular on account of the threaded engagement of dose member 34 and rotation member 21. Thus, the rotation member 21 rotates in the first direction—counter-clockwise as seen from the proximal end of the rotation member—with respect to the housing. Rotation member 21 also rotates with respect to the drive member 20 and to the stop member 26. The drive member 20 is prevented from rotating in the first direction by interaction with the stop member 26, e.g. by interlocking of toothings 27 and 28. As the piston rod 12 is coupled to the drive member 20 and rotation in the first direction of the drive member would cause the piston rod to travel in the proximal direction, the piston rod 12 is prevented from being driven in the proximal direction by interaction of stop member 26 and drive member 20. By preventing the piston rod 12 from moving during dose setting dose accuracy can be increased.

When the rotation member 21 rotates in the first direction, the ramps of the teeth of toothing 23 of rotation member 21 slide along the ramps of the teeth of toothing 22. Thus, a tooth of the rotation member may index around the rotation axis until the tooth engages one of the next teeth of toothing 22 of drive member 20. The teeth of rotation member 21 slide along the ramps of the teeth of drive member 20. During this movement, drive member 20 and, in particular, stop member 26 are displaced along the rotation axis with respect to piston rod 12 and housing by a distance determined by, preferably equal to, the depth of a tooth of toothing 22, before a tooth of toothing 23 (totally) disengages that tooth of toothing 22. Afterwards, the tooth of the rotation member 21 engages the next tooth of toothing 22 and the force provided by resilient member 31 moves drive member 20 and, in particular, stop member 26 back along the rotation axis into the axial start position. An according movement of stop member and drive member in the distal direction and back into the proximal direction is indicated by double arrow 45 in FIGS. 2 and 3.

A tooth of the rotation member which engages the next tooth of the drive member may cause an audible and/or tactile feedback to the user.

The drive mechanism is suitable for a fixed dose device or a user settable dose device. The size of the fixed dose of drug which is to delivered or the increments in which a user-settable dose may be varied by a user are preferably determined by the distribution of the teeth of the respective toothings in the drive member, rotation member and stop member. The rotation member may be rotated over more than one teeth (dose increment) of the drive member for a user-settable dose device and over one teeth (only) for a fixed dose device. The number of teeth in the drive member 20 over which the rotation member 21 rotates during dose setting determines the size of the dose which is actually delivered. The dose member and the rotation member may be adapted to one another such that the rotation member may rotate only by one tooth for a fixed dose device and by more than one tooth for a variable dose device.

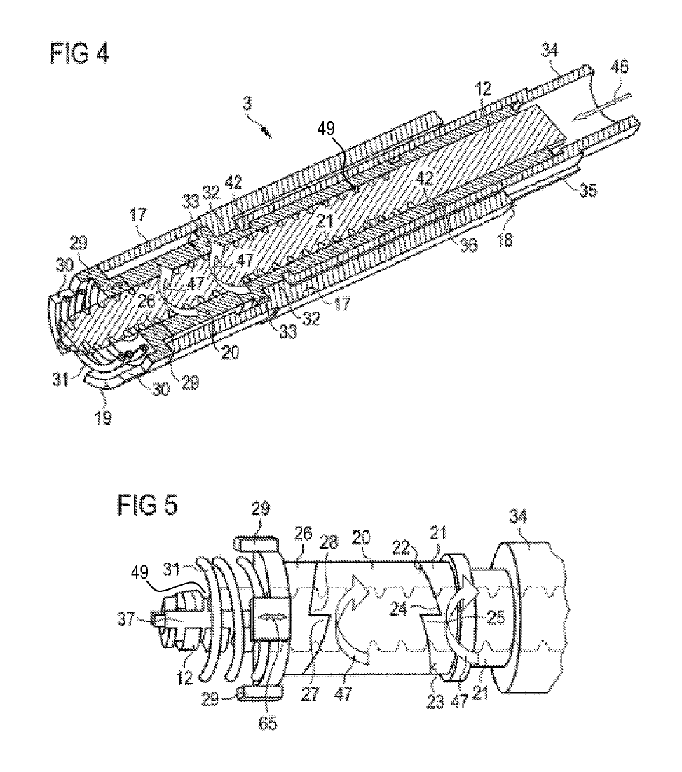

After the dose has been set, the dose part 16 and with it the dose member 34 is moved (pushed) by the user in the distal direction with respect to housing part 17 (arrow 46; cf. FIGS. 4, 5, 8 and 9). Thus, the dose member 34 is moved in the distal direction with respect to the housing part 17. The rotation member 21 accordingly rotates in the second direction, which is opposite to the first direction, with respect to the housing (arrow 47, cf. FIGS. 4 to 9). Drive member 20 follows rotational movement of the rotation member in the second direction. Rotational movement of the drive member 20 in the second direction is converted into rotational movement of the piston rod 12 in the second direction, which movement, in turn, is converted into movement of the piston rod 12 in the distal direction. Accordingly, the piston 10 of FIG. 1 may be displaced in the distal direction with respect to the cartridge 4 and a dose of drug 5 is dispensed from the cartridge the amount of which corresponds to the previously set dose.

During dose delivery, toothings 22 and 23 interlock and ramps of the teeth of toothing 28 of the drive member 20 slide along ramps of the teeth of toothing 27 of stop member 26. This movement is similarly as described above for the relative rotational movement of rotation member and drive member with opposite rotation direction. The stop member 26 is thereby displaced in the distal direction with respect to the drive member 20 by a distance corresponding to the depth of a tooth of toothing 27 in stop member 26. Resilient member 31 forces the stop member 26 back into the axial starting position, when the next tooth of toothing 28 is engaged by the respective tooth of toothing 27 (double arrow 65).

A tooth of the drive member which engages the next tooth of the stop member may cause an audible and/or tactile feedback to the user.

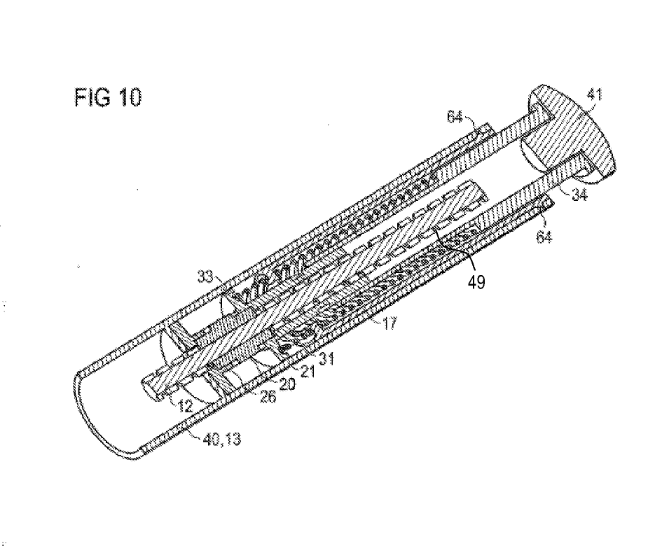

FIG. 10 schematically shows an oblique sectional view of a second embodiment of a drive mechanism. This drive mechanism essentially corresponds to the one described in conjunction with FIGS. 2 to 9. In contrast thereto, the stop member 26 is secured against rotational movement and displacement with respect to the housing (13, 17, 40). Stop member 26 may be integrated in housing part 40 or 17 or an insert thereof. Housing part 40 may be housing 13, for example. Housing part 17 may be inserted and fixed within housing 13. Fixing elements 64 may engage corresponding elements in the housing for fixing the housing part 17 to housing part 40.

In order to compensate for the relative axial displacement between rotation member 21, drive member 20 and stop member 26, when the respective parts rotate with respect to one another, the rotation member 21 is movable with respect to the housing. In order to keep stop member 26 and rotation member 21 in, preferably permanent, abutment with drive member 20 during drug delivery operation of the drive mechanism, resilient member 31 exerts a force on the rotation member 21, preferably on protruding member 33 thereof which presses rotation member and drive member 20 towards stop member 26. Resilient member 31 may be arranged at that side of the drive member which faces away from the stop member, e.g. its proximal side. Resilient member may abut the proximal face of protruding member 33. Support member 32 can thus be dispensed with. The distal end face of housing part 17 may act as an abutment surface for the resilient member 31.

However, when the elements are arranged as shown in FIG. 10, axial movement of the rotation member, which may occur correspondingly to the axial movement of the stop member in the previous embodiment, may be transferred to the dose part 16 and thereby to the user. This movement of an external part might be irritating for a user.

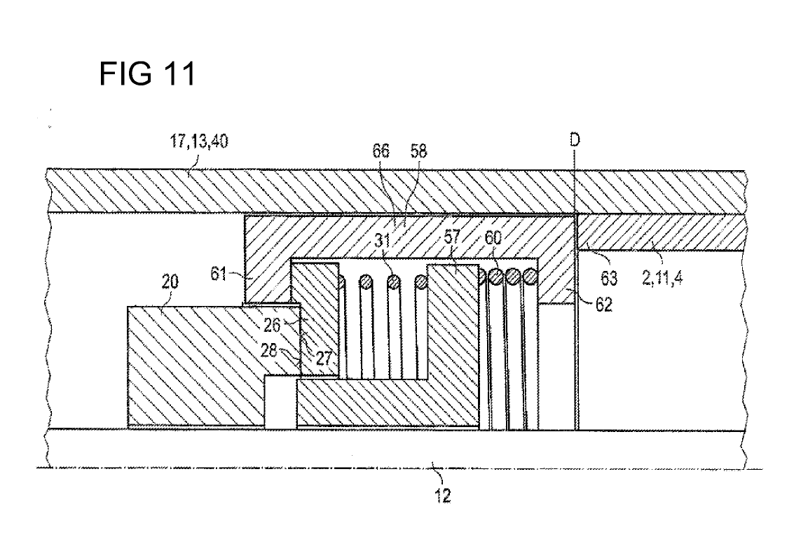

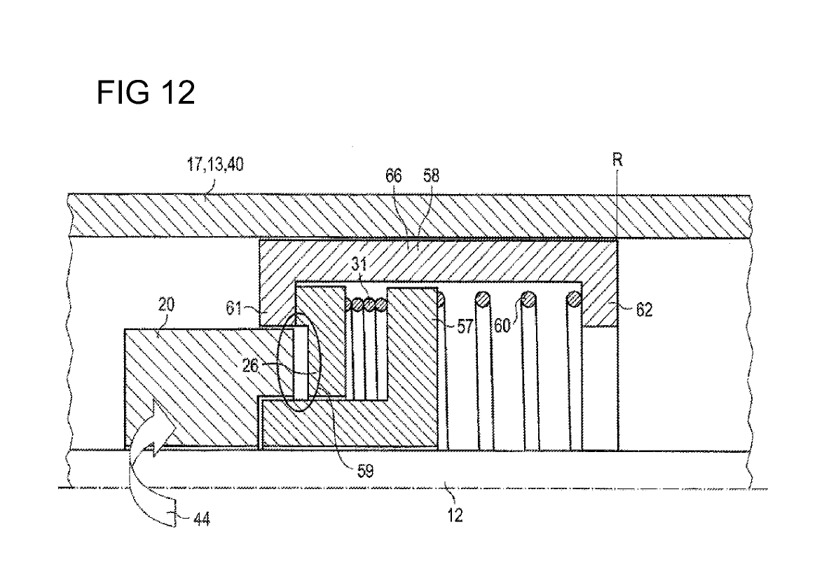

FIG. 11 shows a schematic sectional view of a part of a resettable drive mechanism according to an embodiment in a delivery state. FIG. 12 shows the resettable drive mechanism of FIG. 11 in a reset state.

The drive mechanism may correspond to the one described in conjunction with FIGS. 2 to 9. However, a reset mechanism for a drive mechanism as it is described in more detail below may also be provided for in the remaining drive mechanisms as described above.

The drive mechanism described in conjunction with FIGS. 11 and 12 is a resettable drive mechanism. For this purpose, the drive mechanism comprises a reset mechanism. The reset mechanism may be switched between a reset position and a delivery position.

In contrast to the drive mechanism described in conjunction with the previous figures, the rotation member 21 is not shown in FIGS. 11 and 12. However, a rotation member may nevertheless be provided. FIGS. 11 and 12 only show a half of a section through the drive mechanism. The additional cut was made along piston rod 12. In contrast to the previous embodiments, the distal direction is to the right and the proximal direction is to the left in FIGS. 11 and 12.

As shown in FIG. 11, in the delivery state, drive member 20 and stop member 26 are engaged with one another such that rotational movement of the drive member 20 with respect to housing 13 in the first direction is prevented and rotation of the drive member 20 in the second direction, opposite to the first direction, is allowed. Toothings 27 and 28 may be provided for this purpose as described further above. Resilient member 31 exerts a force acting in axial direction on stop member 26, said force tending to keep the stop member and the drive member engaged. Resilient member 31 may be arranged to keep stop member in engagement and, in particular, in abutment with drive member 20 in the delivery state. The (biased) resilient member 31 may be supported by and, preferably, bear against bearing member 57. Bearing member may be support 48 of FIG. 6, for example. Bearing member 57 is expediently secured against rotational movement and displacement with respect to housing 13.

Rotation of the drive member 20 in the second direction may cause the piston rod 12 to be displaced in the distal direction with respect to housing 13. The piston rod 13 may rotate and translate in the distal direction with respect to the housing for dose delivery as described in conjunction with FIGS. 2 to 10. The drive member 20 may engage the piston rod 12. The drive member 20 may be splined to the piston rod 12. Preferably, there is no relative rotational movement possible between piston rod 12 and drive member 20. Also, the drive member 20 preferably cannot be rotated in the first direction on account of the (permanent) interlocking of the drive member 20 and the stop member 26 when the reset mechanism is in the delivery state.

Thus, when the drive mechanism is in the delivery state, movement of the piston rod 12 in the proximal direction with respect to housing 13 to a starting position is prevented, because the stop member 26 prevents rotation of the drive member 20 in the first direction and the drive member has to be rotated in the first direction, if the piston rod 12 was to be moved in the proximal direction with respect to the housing 13 into the starting position.

However, after a cartridge 4 has been emptied, i.e. after a distal end position of the piston 10 and, in particular, of the piston rod 12 has been reached, the piston rod has to be moved in the proximal direction back into a proximal starting position in order to allow the drive mechanism to be reused. Expediently, the drive mechanism is configured to be switchable from the delivery state to a reset state. In the reset state, the piston rod 12 may be moved in the proximal direction with respect to the housing, for example by a user screwing and/or pushing the piston rod 12 in the proximal direction.

The drive mechanism comprises a clutch member 58. Clutch member 58 is movable with respect to housing 13, preferably displaceable with respect to the housing, between a delivery position D and a reset position R. The clutch member 58 may be moved back and forth between the delivery position and the reset position. The reset position may be arranged in the distal direction as seen from the delivery position. The clutch member 58 may be a sleeve. Piston rod 12 may extend through clutch member.

In the delivery position, drive member 20 and stop member 26 are engaged. In the reset position, drive member 20 and stop member 26 are disengaged (cf. the encircled region 59 in FIG. 12). Thus, when the clutch member 58 is in the reset position, the drive member may be rotated in the first direction with respect to the housing 13 without the stop member 26 preventing the rotation. Consequently, the piston rod 12 may be moved in the proximal direction, e.g. by rotation with respect to the housing and on account of a threaded engagement to the housing, due to the drive member 20 and the stop member 26 being disengaged.

The clutch member 58 may comprise a protrusion 61. Protrusion 61 may protrude radially and preferably inwardly from a base portion 66 of the clutch member 58. The base portion may extend in the axial direction. Protrusion 61 may be arranged to move the drive member 20 and the stop member 26 out of engagement when the clutch member is moved towards reset position R. Protrusion 61 may be provided at or near the proximal end of the clutch member 58. A distal end face of protrusion 61 of clutch member 58 may be arranged to couple to and preferably to abut a proximal face of stop member 26.

The reset mechanism furthermore comprises a clutch resilient member 60, for example a clutch spring member, like a coil spring and/or a compression spring, for example.

The clutch member 58 may extend along drive member 20, stop member 26, resilient member 31, bearing member 57 and/or clutch resilient member 60. The clutch member 58 may be rigid. The clutch member 58 may have a constant length.

Clutch resilient member 60 may be biased when the clutch member 58 is in the delivery position. Biased clutch resilient member may exert a force on the clutch member that tends to move the clutch member in the reset position. Clutch resilient member 60 may bear on bearing member 57, in particular on a distal face thereof.

Clutch member 58 may comprise a (additional) protrusion 62. Protrusion 62 may protrude radially and preferably inwardly from the base portion 66 of the clutch member 58. Protrusion 62 may be arranged in the region of the distal end of the clutch member 58. Protrusion 62 may be arranged to be abuttable by and is preferably abutted by clutch resilient member 60. Clutch resilient member 60 may be supported by and, in particular, bear on a proximal face of protrusion 62.

The clutch resilient member 60 is arranged to exert a force on the clutch member 58 which force tends to move the clutch member 58 in the reset position R. When the drive mechanism is in the delivery state, this force is counteracted by a clutch stop member 63. Accordingly, in the delivery state, clutch member 58 may be held in the delivery position by the clutch stop member 63.

In the delivery state, clutch stop member 63 is preferably secured against displacement with respect to the housing 13. Clutch stop member 63 may be arranged to abut clutch member 58. A proximal end face of the clutch stop member 63 may abut a distal end face of the clutch member 58 in the delivery state.

For resetting the device, the clutch stop member 63 may be moved, for example removed, so as to allow the clutch member to move into the reset position. Thereupon, biased clutch resilient member 60 which exerts the force, which is no longer compensated by clutch stop member, on clutch member 58. The force automatically tends to move clutch member 58 in the reset position R. The clutch member 58 may abut stop member 26. Stop member 26 may tend to follow movement of the clutch member towards the reset position R.

In order to get into reset position the force exerted by the resilient member 31 on the stop member 26, which force tends to hold drive member 20 and stop member 26 in engagement, has to be overcome. Thus, the force moving the clutch member 58 towards the reset position 58 has to be greater than the force exerted by the resilient member 31. The force for moving and, in particular, holding the clutch member 58 in reset position R may be provided for by clutch resilient member 60. It is expedient for the resilient member 31 and the clutch resilient member 60 to be embodied as a spring member, respectively. Clutch resilient member 60, in this case, preferably has a spring strength greater than the one of resilient member 31 in order to overcome the force exerted by resilient member 31.

The clutch stop member 63 is expediently formed in the cartridge unit, for example, by the cartridge 4 or the cartridge retaining member 11. Thus, if the cartridge unit is detached from the housing 13 for replacing an empty cartridge, the clutch member 58 is moved, preferably automatically, towards and into the reset position and preferably held in the reset position.

The distance by which the clutch member 58 moves with respect to the housing 13 when moving from delivery position into reset position is preferably chosen to be great enough to disengage toothings 27 and 28.

The clutch member 58 is expediently secured to the drive mechanism in order to avoid the clutch member falling out of the housing. For this purpose, the clutch member may abut a proximal face of the stop member 26.

The clutch member 58 may be axially guided with respect to the housing 13 when it is moved from the delivery position D into the reset position R and preferably also when it is moved from the delivery position back into the reset position after the reset has been completed. The clutch member 58 may be secured against rotational movement with respect to the housing 13.

As shown in FIG. 12, when the clutch member 58 is in reset position R, the drive mechanism is in the reset state and the piston rod 12 may be moved in the proximal direction with respect to the housing from a distal end position back into a proximal starting position. When a new cartridge 4 is attached to the housing 13, after the piston rod 12 was moved back into starting position, clutch member 58 may be moved into the distal direction back into delivery position together with the cartridge 4 and, if present, the cartridge retaining member 11, thereby moving drive member 20 and stop member 26 again into engagement.

Accordingly, the drug delivery device may be reused. As an element of the cartridge unit like cartridge 4 or cartridge retaining member 11 may serve as the clutch stop member 63, the reset mechanism may automatically and, in particular (purely) mechanically, decouple stop member 26 and drive member 20, when the cartridge unit 2 is detached from the drive unit 3 (cf. FIG. 1). Thus, the only action required by a user is to move, e.g. screw and/or push, the piston rod 12 back into the starting position before a new cartridge unit 2 may be attached to the drive unit 3. The drive mechanism is thus easily reusable.

The reset mechanism described herein above may be implemented easily and requires only a small amount of additional parts such as compared to the corresponding non-resettable drive mechanism. In particular, such as compared to the first embodiment, only two additional parts—clutch member and clutch resilient member—are required for the automatic reset mechanism.

As the reset mechanism may be an automatic one, no external action is required for disengaging stop member and drive member. Thus, the clutch member may be retained in the housing and, in particular, inaccessible from the outside.

Of course, the reset mechanism may be implemented as a manual, non-automatic mechanism. It is expedient, in this case, to configure the movement of the clutch member to be externally actuable.

In contrast to the situation depicted in FIGS. 11 and 12, the clutch member 58 may be (partly) arranged outside of the housing. The housing may be provided with one or more openings through which the clutch member may extend from the outside to the inside of the housing. This is particularly expedient for a non-automatic reset mechanism.

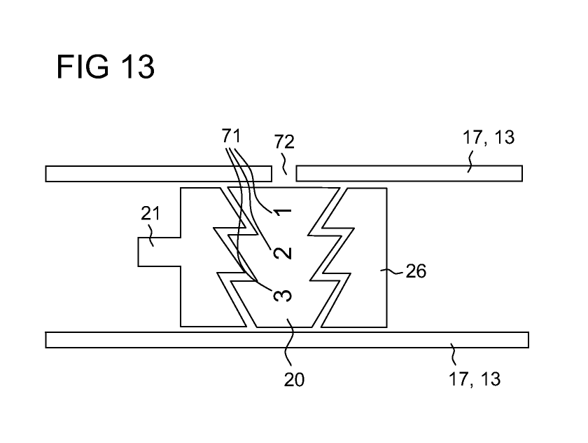

FIG. 13 shows parts of another exemplary embodiment of a drug delivery device on the basis of a schematic sectional view.

Essentially, the embodiment shown in FIG. 13 corresponds to the ones described previously in connection with FIGS. 1 to 12. However, elements of the previous figures, which are not necessary to illustrate the embodiment in FIG. 13, are not explicitly shown. In particular, the resilient member 31 and the piston rod 12 are not explicitly illustrated in FIG. 13. FIG. 13 shows the drive member 20, which is arranged between rotation member 21 and stop member 26. Stop member 26 prevents rotation of the drive member when the rotation member 21 is rotated for dose setting. Rotation member 21 rotates with respect to the drive member 20 during dose setting and carries the drive member 20 with it when it is rotated for dispensing a dose in the opposite direction. The clutch mechanisms provided for this purpose operate as described previously. Accordingly, all features described previously for the drive mechanism or the resettable drive mechanism and the drug delivery device apply also for the embodiment of FIG. 13.

In contrast to the previous embodiments, the drive member 20 is provided with a plurality of indication elements 71. The indication elements may be formed unitarily with the drive member or may be applied separately on the drive member, for example by printing. The indication elements may comprise numbers.

The indication elements 71 are provided on an outer surface of the drive member. The outer surface of the drive member is visible from the outside of the housing 13 or the housing part 17. Accordingly, a user may view the outer surface of the drive member from outside of the device. For this purpose, a window 72 is provided in the housing 13 and/or housing part 17. The window may be a window aperture which may or may not be provided with a window part which covers the aperture in the housing. Alternatively, housing 13 and/or housing part 17 may be transparent so as to allow the outer surface of the drive member 20 to be viewed through the housing 13 from outside of the housing.

One of the indication elements may be displayed through the housing so as to indicate dose-related information about the drug contained in the cartridge (not explicitly illustrated). The displayed indication element can be framed by the window or be distinguished from the remaining indication elements by a marker, for example an arrow or a triangle provided on the outside of the transparent housing, or other distinguishing means. Accordingly, the drive member 20 may be simultaneously used for indicating dose-related information to the user and to drive movement of the piston rod for dispensing a dose of drug. The drive member 20 may act as a dose counter, which counts the number of available or dispensed doses in a fixed dose device.

As the drive member is immediately coupled to the piston rod (not explicitly illustrated in FIG. 13) by a splined connection, the rotation angle, by which the drive member is rotated permits to gather reliable information about the position of the piston rod with respect to the housing 13. Accordingly, this permits to gather reliable information about the quantity of drug still present in the cartridge or the quantity of drug already dispensed from the cartridge.

The distance between two indication elements 71 corresponds to or is determined by the rotation angle by which the drive member 21 is rotated for setting and/or dispensing a dose of the drug. Accordingly, the distance between two indication elements corresponds to or is determined by the rotation angle by which the drive member is rotated for dispensing the dose. Preferably, one indication element is assigned to each tooth of the toothing provided on the drive member for the uni-directional clutch which couples the drive member 20 to the stop member 26 and/or to the rotation member 21.

The distance between indication elements 71 reflects the angle of rotation by which the drive member 20 and the rotation member 21 are rotated and is determined by the size of the outer diameter of the drive member 20.

When the rotation member 21 is rotated, the drive member 20 may be axially displaced with respect to the housing in a limited fashion as described previously. This may result in a limited axial movement of the indication elements including, of course, the displayed indication element. Expediently, the size of the window 72 is chosen so as to keep the indication element which is to be currently displayed visible even if the drive member is axially displaced. However, in a resting state, e.g. when the rotation member 21 is not rotated, the displayed indication element is preferably always visible for a user through the window 72.

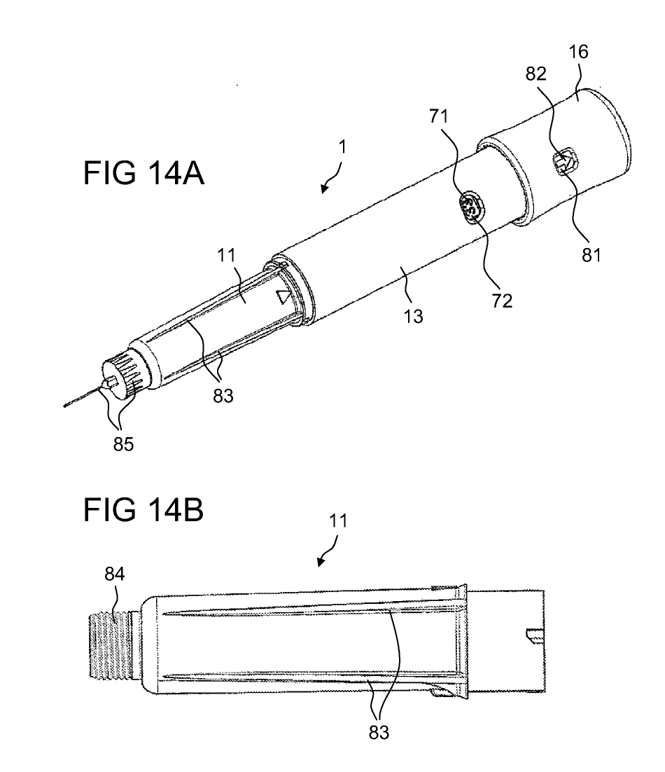

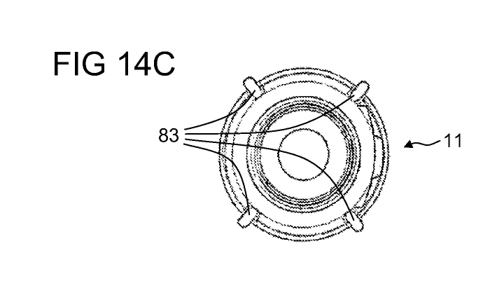

FIGS. 14A to 14C show another exemplary embodiment of a drug delivery device 1 on the basis of a schematic perspective view in FIG. 14A, a schematic side view on the cartridge retaining member in FIG. 14B and a schematic sectional view through the cartridge retaining member of FIG. 14B in FIG. 14C.

The drug delivery device 1 which is depicted in FIG. 14A largely corresponds to the one depicted in FIG. 1, expect for the window 72 in the housing 13 and a window 81 in the dose part 16 which reveals an operating symbol 82 which may indicate to the user that the dose part 16 has to be pulled away from the housing 13 for setting a dose which is to be dispensed subsequently. Furthermore, dose part 16 surrounds the housing 16. Operating symbol 82 may be applied on an outer surface of the housing 13.

The drive mechanism and the remaining elements described in conjunction with FIGS. 1 to 13 may also be present in the embodiment described in connection with FIGS. 14A to 14C.

In contrast to the previously described embodiments, the cartridge retaining member 11 or cartridge holder comprises one or a plurality of protruding elements 83. The respective protruding element protrudes radially from the cartridge holder 11 and is longitudinally oriented. The radial extension may decrease in distal direction as seen along the protruding element. The respective protruding element is formed rib-like. The protruding elements 83 may facilitate detaching of the cartridge retaining member 11 from the housing 13 for replacing an empty cartridge in the cartridge retaining member with a replacement cartridge.

Such as compared to a smooth surface of the cartridge holder, the protruding elements which may serve as grip splines, may facilitate the detaching action of the cartridge holder from the housing which has to be performed by a user. The user may transfer the force required for detaching or re-attaching the cartridge holder to the holder more easily, if the protruding elements are provided. Users of drug delivery devices, in particular users suffering from diabetes or from other illnesses which require a regular, e.g. daily, treatment, often have impaired motor functions or limited dexterity. The protruding elements assist the user to get a firm grip of the cartridge holder. Accordingly, the detaching action is facilitated by providing the protruding elements.

The protruding elements may—additionally or alternatively to their function as grip splines—act as lead which may guide attachment of a cap (not explicitly illustrated) to the housing 13.

Also, as depicted in FIG. 14B, the cartridge holder comprises fixing means 84, for example a thread, which allows a needle unit 85 to be releasably secured to the cartridge retaining member 11.

Of course, the invention is not restricted by the embodiments described above.

1-15. (canceled)

16. A drug delivery device, comprising:
a housing,
a drive member which is movably retained within the housing,
a piston rod which is operatively coupled to the drive member, wherein
the drug delivery device is configured to convert movement of the drive member with respect to the housing into movement of the piston rod with respect to the drive member,
wherein a surface of the drive member is provided with one or a plurality of indication elements, and
wherein the drug delivery device is adapted to display at least one of the indication elements through the housing.

17. The device of claim 16, wherein the drive member engages the piston rod.

18. The device of claim 16, wherein the drive member is splined to the piston rod.

19. The device of claim 16, wherein the device is a fixed dose device for dispensing a plurality of predefined doses of drug.

20. The device of claim 16, wherein the displayed indication element provides information about the number of doses of drug dispensed from the device or remaining to be dispensed from the device.

21. The device of claim 16, wherein the drive member is configured to be rotatable with respect to the housing for dispensing a dose of drug, wherein the rotation angle by which the drive member is rotated for dispensing the dose corresponds to or is determined by the distance between adjacent indication elements.

22. The device of claim 16, which is configured such that the displayed indication element changes when a dose of drug is dispensed from the device.

23. The device of claim 16, wherein the housing has a proximal end and a distal end, and the device is configured to convert rotational movement of the drive member into distal movement of the piston rod for dispensing a dose of drug.

24. The device of claim 16, wherein, in a resting state of the device in which the device is not in use for setting or dispensing a dose, the drive member is secured against axial displacement with respect to the housing and a window is provided in the housing which frames the displayed indication element.

25. The device of claim 16, which comprises a rotation member which is adapted to be rotated with respect to the drive member in a first direction for setting a dose of drug and to be rotated in a second direction with respect to the housing for dispensing the set dose of drug.

26. The device of claim 25, wherein the rotation angle by which the rotation member is rotated for dose setting and/or for dose dispensing corresponds to or is determined by the distance between adjacent indication elements.

27. The device of claim 25, wherein the drive member is coupled to the rotation member by a uni-directional clutch mechanism which is, when the clutch mechanism is engaged, operative to permit relative rotational movement of the rotation member with respect to the drive member when the rotation member is rotated in the first direction and to prevent relative rotational movement of the rotation member with respect to the drive member when the rotation member is rotated in the second direction.

28. The device of claim 25, which comprises a stop member, wherein the stop member is coupled to the drive member by a uni-directional clutch mechanism which is, when the clutch mechanism is engaged, operative to prevent relative rotational movement of the drive member with ct to the stop member when the rotation member is rotated in the first direction and to permit relative rotational movement of the drive member with respect to the stop member in the second direction.

29. The device of claim 28, wherein the drive member is arranged between the stop member and the rotation member, and wherein a resilient member is provided which keeps the uni-directional clutch mechanisms permanently engaged when the device is operated for setting and dispensing a dose of drug.

30. Using a single member in a drug delivery device for driving movement of a piston rod of the device and for displaying dose related information to a user of the device.
